(19) United States
(12) Patent Application Publication
(10) Pub. No.: US 2025/0228726 A1
(43) Pub. Date: Jul. 17, 2025

(54) MENTUM SECUREMENT DEVICE AND METHOD OF USING THE SAME

(71) Applicant: JD3 Labs Inc, St Paul, TX (US)

(72) Inventors: James Dongbo Tang, St Paul, TX (US); John Stephen Crombie, East Hnover, NJ (US)

(21) Appl. No.: 19/025,247

(22) Filed: Jan. 16, 2025

Related U.S. Application Data
(60) Provisional application No. 63/621,257, filed on Jan. 16, 2024.

(51) Int. Cl. A61G 13/12; A61F 5/37

(52) U.S. Cl. CPC A61G 13/1215; A61F 5/3707

(57) ABSTRACT

A mentum securement device and method of using the same for securement of a mentum of a patient at an angle such that obstruction of an airway of the patient is prevented. The mentum device includes a mentum strap and a pair of securement straps. The mentum strap is configured to be positioned on a mentum of a patient undergoing anesthesia. The pair of securement straps are attached to distal portions of the mentum strap and have a distal portion securable to a respective anchor point. The mentum securement device is configured to position the mentum in an extended state where the mentum is at an extended angle relative to a neck of the patient.

### RELATED APPLICATION

This application claims priority to and the benefit of U.S. Provisional Patent Application No. 63/621,257, filed Jan. 16, 2024 and entitled MENTUM SECUREMENT DEVICE AND METHOD OF USING THE SAME, the contents of which are incorporated herein by reference in their entirety.

### FIELD

The present disclosure generally relates to anesthesia devices. In embodiments, the present disclosure relates to a mentum securement device and methods of using the same for securement of a mentum of a patient at an angle.

### BACKGROUND

Anesthesia may cause airway obstruction of a patient due to relaxation of the larynx and associated upper airway muscles and structures, due to suppression of protective arousal responses, and due to decreases in respiratory reserve resulting from relaxation of the lung. Patients undergoing anesthesia may undergo orotracheal intubation to maintain an open airway. Intubation and subsequent extubating may cause minor injury such as dysphonia, aphonia and dysphagia. More severe intubation-related laryngeal trauma may include, for example, scarring of the larynx and chronic laryngeal stenosis, all of which may be associated with significant morbidity and can be life-threatening.

Certain medical procedures do not require intubation, and the patient may merely undergo sedation anesthesia. Generally, the need for intubation is determined by one or more of: the duration of the medical procedure, degree of painful stimuli, the use of muscle relaxants during the procedure and the type of procedure, to name a few. By way of example, certain surgeries result in significant blood loss and/or significant changes in the patient's breathing, blood pressure or heart rate, and therefore require general anesthesia and intubation. However, the risk of airway obstruction is still present even under sedation anesthesia.

Practitioners administering the anesthesia monitor the vital signs of the patient (such as heartbeat, breathing rate, temperature, and blood pressure) while controlling the delivery of anesthesia to the patient. In instances where an airway obstruction of the patient is occurring, the practitioner may have to intervene while continuing to monitor the breathing quality of the patient such that the airway remains open. This may last for a few seconds to minutes, or up to the remaining duration of the medical procedure. The intervention may be burdensome for the practitioner, and interferes with the monitoring of anesthesia delivery to the patient.

### SUMMARY

An object of the present invention is to provide a mentum securement device for passively managing, reducing, and potentially preventing obstruction of the airway of a patient during non-intubated anesthesia enabling the practitioner freedom from airway management tasks so that vital signs can be adequately monitored and delivery of anesthesia to the patient can be controlled.

In embodiments, a mentum securement device is disclosed. The mentum securement device comprises: a mentum strap defined by opposing distal portions, a superior end and an opposing inferior end, and a first surface configured to be positioned on a mentum of a patient undergoing anesthesia; and, a pair of securement straps each having a proximal portion attached to a respective opposing distal portion of the mentum strap and a distal portion securable to a respective anchor point, wherein the mentum strap and the pair of securement straps are configured to position the mentum of the patient to an extended state, wherein in the extended state the mentum of the patient is at an extended angle relative to a neck of the patient, wherein in a resting state the mentum of the patient is at a resting angle relative to a neck of the patient, wherein the extended angle is greater than the resting angle.

In embodiments, an airway passage of the patient is unobstructed in the extended state.

In embodiments, a tilt angle is defined as the difference between the extended angle and the resting angle.

In embodiments, the tilt angle in the extended state is in the range of 10° to 30°.

In embodiments, in use during an anesthetic procedure, securement of the mentum in the extended state prevents obstruction of an airway passage of the patient.

In embodiments, the tilt angle is set by adjustment of a respective length of the pair of securement straps.

In embodiments, each of the pair of securement straps comprise a webbing and buckle configuration to adjust the respective lengths of the pair of securement straps.

In embodiments, the pair of securement straps further comprise a hook-and-loop portion at the distal portion configured for adjustment of the respective lengths of the pair of securement straps by interlocking the hook-and-loop portion at one or more positions along lengths of the pair of securement straps.

In embodiments, each of the distal portions of the pair of securement straps comprise a hook-and-loop portion configured to removably secure the distal portions to respective anchor points in a hook-and-loop securement.

In embodiments, the respective anchor point is a portion of a siderail of a surgical stretcher.

In embodiments, respective anchor point is a tie-down d-ring of a surgical stretcher.

In embodiments, each of the distal portions of the pair of securement straps comprise a tacky first surface to removably secure the distal portions to respective anchor points.

In embodiments, each of the pair of securement straps are configured to fail upon application of a tension force.

In embodiments, the tension force is in the range of 0.1 to 10 lbs-ft.

In embodiments, the tension force is selected such that the tilt angle cannot exceed the range of 10° to 30° relative to a resting state of the mentum.

In embodiments, a hook-and-loop portion of the pair of securement straps is configured to detach upon application of the tension force.

In embodiments, the mentum strap is comprised of an elastomeric material.

In embodiments, the elastomeric material comprises a material selected from the group consisting of: medical grade silicone, heat cured rubber (HSR) elastomer, high temperature vulcanizing silicone rubber, and liquid silicone rubber (LSR) elastomer.

In embodiments, the elastomeric material has a tensile strength value.

In embodiments, the elastomeric material has a Young's modulus value.

In embodiments, at least a portion of the first surface comprises a tacky surface configured to be removably adhered to the mentum of the patient.

In embodiments, the first surface comprises an adhesive coating.

In embodiments, the mentum strap is comprised of a tacky elastomeric material.

In embodiments, the elastomeric material has a tackiness index in the range.

In embodiments, the mentum strap further comprises a plurality of perforations.

In embodiments, a method of preventing obstruction of an airway passage of a patient undergoing an anesthetic procedure is disclosed. The method comprises: evaluating breathing quality of the patient when a mentum of the patient is in a resting state defined by a resting angle of the mentum of the patient relative to a neck of the patient; positioning a mentum strap of a securement device on the mentum of the patient, the mentum securement device comprising a pair of securement straps each having a proximal portion attached to a respective opposing distal portion of the mentum strap; securing distal portions of the pair of securement straps to respective anchor points of a surgical stretcher; and, adjusting lengths of the pair of securement straps so that the mentum of the patient is moved from the resting state to an extended state, wherein in the extended state the mentum of the patient is at an extended angle relative to a neck of the patient and the patient no longer has obstructed breathing, wherein the extended angle is greater than the resting angle.

In embodiments, the breathing quality of the patient is evaluated by detecting one or more of labored breathing, obstructed breathing, and snoring.

In embodiments, the breathing quality of the patient is evaluated by monitoring carbon dioxide readings from a gas sampling device attached to the patient.

A mentum securement device according to an exemplary embodiment of the present invention comprises: a mentum strap defined by opposing distal portions, a superior end and an opposing inferior end, and a first surface configured to be positioned on and in facing relation to a mentum of a patient undergoing anesthesia; and two or more securement straps each having a proximal portion attached to a respective opposing distal portion of the mentum strap and a distal portion configured for securement to a an anchor point, wherein the mentum strap and the two or more securement straps are configured to position the mentum of the patient to an extended state, wherein in the extended state the mentum of the patient is at an extended angle relative to a neck of the patient, wherein in a resting state the mentum of the patient is at a resting angle relative to a neck of the patient, wherein the extended angle is greater than the resting angle.

In exemplary embodiments, an airway passage of the patient is unobstructed in the extended state.

In exemplary embodiments, a tilt angle is defined as the difference between the extended angle and the resting angle, and the tilt angle in the extended state is in the range of 100 to 300.

In exemplary embodiments, the tilt angle is set by adjustment of a respective length of the pair of securement straps.

In exemplary embodiments, each of the pair of securement straps comprise a webbing and buckle configuration to adjust the respective lengths of the pair of securement straps.

In exemplary embodiments, the pair of securement straps further comprise a hook-and-loop portion at the distal portion configured for adjustment of the respective lengths of the pair of securement straps by interlocking the hook-and-loop portion at one or more positions along lengths of the pair of securement straps.

In exemplary embodiments, each of the distal portions of the pair of securement straps comprise a hook-and-loop portion configured to removably secure the distal portions to respective anchor points in a hook-and-loop securement.

In exemplary embodiments, the respective anchor point is a portion of a siderail of a surgical stretcher.

In exemplary embodiments, the respective anchor point is a tie-down d-ring of a surgical stretcher.

In exemplary embodiments, each of the distal portions of the pair of securement straps comprise a tacky first surface to removably secure the distal portions to respective anchor points.

In exemplary embodiments, the two or more securement straps are configured to fail upon application of a predetermined tension force.

In exemplary embodiments, the tension force is in the range of 0.1 to 10 lbs-ft.

In exemplary embodiments, the tension force is selected such that the tilt angle cannot exceed the range of 10° to 30° relative to a resting state of the mentum.

In exemplary embodiments, a hook-and-loop portion of the pair of securement straps is configured to detach upon application of the tension force.

In exemplary embodiments, the mentum strap is comprised of an elastomeric material.

In exemplary embodiments, the elastomeric material comprises a material selected from the group consisting of: medical grade silicone, heat cured rubber (HSR) elastomer, high temperature vulcanizing silicone rubber, and liquid silicone rubber (LSR) elastomer.

In exemplary embodiments, the elastomeric material has a tensile strength value in the range of 0.5 to 15 MPa.

In exemplary embodiments, the elastomeric material has an Young's modulus value in the range of 0.0005 to 0.1 GPa.

In exemplary embodiments, at least a portion of the first surface comprises a tacky surface configured to be removably adhered to the mentum of the patient.

In exemplary embodiments, the first surface comprises an adhesive coating.

In exemplary embodiments, the mentum strap is comprised of a tacky elastomeric material.

In exemplary embodiments, the mentum strap further comprises a plurality of perforations.

In exemplary embodiments, the mentum strap comprises one or more pleats.

In exemplary embodiments, the device further comprises a brace comprising a proximal end portion configured for attachment to the two or more securement straps.

In exemplary embodiments, the brace further comprises a distal end portion configured for attachment to the anchor point.

In exemplary embodiments, the device further comprises a clamping mechanism configured for attachment to the distal end portion of the brace and the anchor point.

In exemplary embodiments, the device further comprises an adjustable tension device disposed between the clamping mechanism and the brace.

In exemplary embodiments, the device further comprises two or more rods each comprising a proximal end portion configured for attachment to a corresponding one of the two or more securement straps and a distal end portion configured for attachment to respective anchor points.

In exemplary embodiments, the two or more rods are adjustable in length.

In exemplary embodiments, an angle at which the two or more rods extend relative to the respective anchor points is adjustable.

According to an exemplary embodiment of the present invention, a method of preventing obstruction of an airway passage of a patient undergoing an anesthetic procedure comprises: evaluating breathing quality of the patient when a mentum of the patient is in a resting state defined by a resting angle of the mentum of the patient relative to a neck of the patient; positioning a mentum strap of a securement device on the mentum of the patient, the mentum securement device comprising two or more securement straps each having a proximal portion attached to a respective opposing distal portion of the mentum strap; securing distal portions of the two or more securement straps to respective anchor points of a surgical stretcher; and adjusting lengths of the two or more securement straps so that the mentum of the patient is moved from the resting state to an extended state, wherein in the extended state the mentum of the patient is at an extended angle relative to a neck of the patient and the patient no longer has obstructed breathing, wherein the extended angle is greater than the resting angle.

In exemplary embodiments, the breathing quality of the patient is evaluated by detecting one or more of labored breathing, obstructed breathing, and snoring,

In exemplary embodiments, the breathing quality of the patient is evaluated by monitoring carbon dioxide readings from a gas sampling device attached to the patient.

A mentum securement device according to an exemplary embodiment of the present invention comprises: a mentum strap defined by opposing distal portions, a superior end and an opposing inferior end, and a first surface configured to be positioned on and in facing relation to a mentum of a patient undergoing anesthesia; and two or more securement straps each having a proximal portion attached to a respective opposing distal portion of the mentum strap and a distal portion; and means for securing the distal portions of the two securement straps to an anchor point, wherein the mentum strap and the two or more securement straps are configured to position the mentum of the patient to an extended state, wherein in the extended state the mentum of the patient is at an extended angle relative to a neck of the patient, wherein in a resting state the mentum of the patient is at a resting angle relative to a neck of the patient, wherein the extended angle is greater than the resting angle.

Other and further objects will be explained hereinafter and more particularly delineated in the appended claims.

### BRIEF DESCRIPTION OF THE DRAWINGS

FIG. 1 is a perspective view of a mentum securement device in accordance with exemplary embodiments of the present disclosure;

FIG. 2 is a front view of a mentum strap according to an exemplary embodiment of the present disclosure;

FIG. 3 is a perspective view of the mentum securement device in accordance with exemplary embodiments of the present disclosure;

FIG. 4 is a perspective view of a mentum securement device according to an exemplary embodiment of the present invention;

FIG. 5 is a perspective view of the mentum securement device secured to a mentum of a patient in accordance with exemplary embodiments of the present disclosure;

FIG. 6A is a side view of the mentum of the patient in a resting state where the mentum securement device is not in use;

FIGS. 6B and 6C are side views of the mentum securement device secured to the mentum of the patient in an extended state in accordance with exemplary embodiments of the present disclosure;

FIG. 7 is a front view of a distal portion of a securement strap in accordance with exemplary embodiments of the present disclosure;

FIG. 8 is a perspective view of the securement strap attached to an anchor point in accordance with exemplary embodiments of the present disclosure; and

FIG. 9A is a perspective view of a mentum securement system according to an exemplary embodiment of the present invention;

FIG. 9B is a detailed perspective view of a portion of a mentum securement system according to an exemplary embodiment of the present invention;

FIG. 10 is a perspective view of a mentum securement system according to an exemplary embodiment of the present invention;

FIG. 11A is a perspective view of a mentum securement system according to an exemplary embodiment of the present invention;

FIG. 11B is a detailed perspective view of a portion of a mentum securement system according to an exemplary embodiment of the present invention;

FIG. 12 is a perspective view of a mentum securement system according to an exemplary embodiment of the present invention;

FIG. 12 illustrates steps for a method of preventing obstruction of an airway passage of a patient undergoing an anesthetic procedure.

In the drawings, exemplary embodiments of the invention are illustrated by way of example, it being expressly understood that the description and drawings are only for the purpose of illustration of exemplary embodiments and are not intended as a definition of the limits of the invention.

### DETAILED DESCRIPTION OF THE EMBODIMENTS

The following description is presented to enable a person of ordinary skill in the art to make and use the invention, and is provided in the context of particular applications and their requirements. Various modifications to the embodiments will be readily apparent to those skilled in the art, and the generic principles defined herein may be applied to other embodiments and applications without departing from the spirit and scope of the invention. In the following description, numerous details are set forth for the purpose of explanation. However, one of ordinary skill in the art will realize that the invention may be practiced without the use of these specific details. In other instances, well-known structures and devices are shown in block diagram form in order not to obscure the description of the invention with unnecessary detail. Thus, the present disclosure is not intended to be limited to the embodiments shown, but is to be accorded the widest scope consistent with the principles and features disclosed herein.

There is an increased risk of an airway obstruction of a patient undergoing non-intubated anesthesia relative to intubated anesthesia. In intubated anesthesia, the endotracheal tube inserted through the larynx of the patient maintains the airway open. In exemplary embodiments, the present invention relates to a mentum securement device and methods of using the same for securing at an angle a mentum of a patient undergoing non-intubated anesthesia or other medical procedure at an angle such that obstruction of an airway of the patient is prevented. The mentum device includes a mentum strap and one or more (e.g., a pair) of securement straps. The mentum strap is defined by opposing distal portions, a superior end and an opposing inferior end, and a first surface configured to be positioned on a mentum of a patient undergoing anesthesia. The securement straps each having a proximal portion attached directly or indirectly to a respective opposing distal portion of the mentum strap and a distal portion securable to a respective anchor point. The mentum strap and the securement straps are configured to position the mentum of the patient to an extended state. In the extended state the mentum of the patient is at an extended angle relative to a neck of the patient. In a resting state the mentum of the patient is at a resting angle relative to a neck of the patient, and the extended angle is greater than the resting angle. A tilt angle is defined as the difference between the extended angle and the resting angle.

As used herein, the terms “lateral,” “medial,” “superior,” “inferior,” “distal” and “proximal” are relative positional terms each having their respective plain meaning, and are not intended to be limiting.

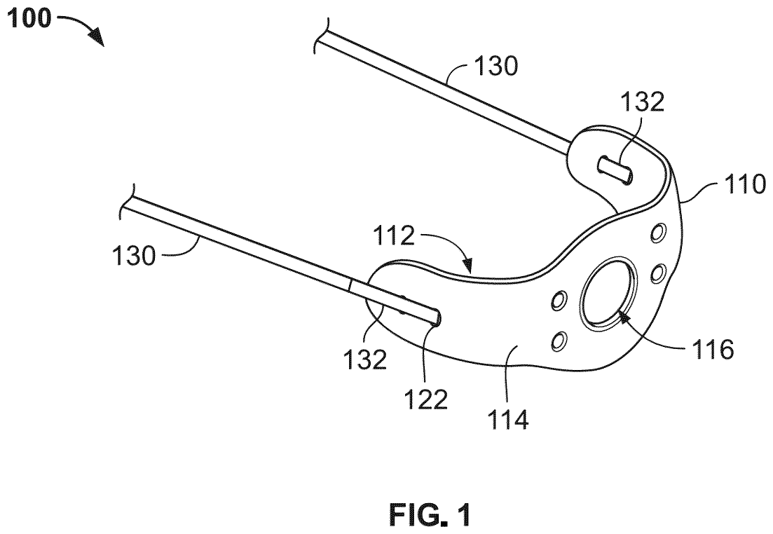

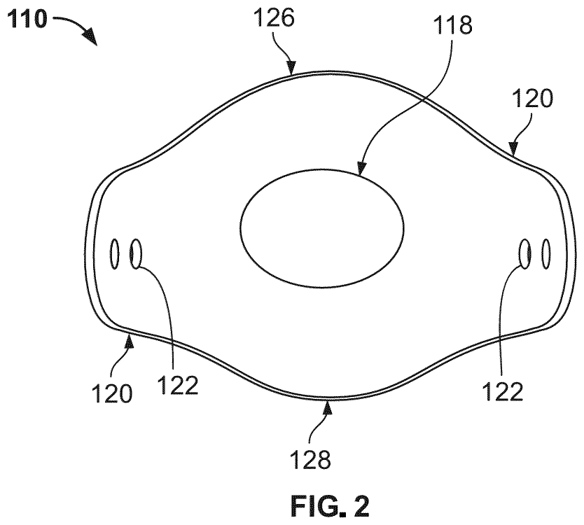

FIG. 1 is a perspective view of a mentum securement device 100 in accordance with exemplary embodiments of the present disclosure. FIG. 2 is a front view of a mentum strap 110 according to an exemplary embodiment of the present disclosure. With reference to FIGS. 1 and 2, the mentum securement device 100 includes a mentum strap 110 and a pair of securement straps 130 attached to the mentum strap 110. In embodiments, the mentum strap 110 has a shape, such as, for example, generally oval or a rectangular, to name just two. The mentum strap 110 has a first surface 112 and a second surface 114, and is defined by opposing distal portions 120, a superior end 126 and an opposing inferior end 128. In embodiments, the superior end 126 forms an extending portion of the mentum strap 110. The first surface 112 is configured to be positioned on and in a directly facing relation to a mentum of a patient undergoing anesthesia as explained in further detail below. In embodiments, the mentum strap 110 is comprised of an elastomeric material. In embodiments, at least a portion of the first surface 112 comprises a tacky surface configured to be removably adhered to the mentum of the patient. In embodiments, the first surface 112 comprises an adhesive coating. The tack surface or adhesive coating enables the mentum strap 110 to remain on the mentum of the patient at least temporarily as a practitioner is attaching the pair of securement straps 130 to anchor points as explained in further detail below. As used herein, the term “tacky surface” generally refers to a surface having adequate adhesion such that the mentum strap 110 may remain on the mentum of the patient, such that the tacky surface does not leave an adhesive residue as the mentum strap 110 is removed from the mentum of the patient.

In embodiments, the elastomeric material may include medical grade silicone, heat cured rubber (HSR) elastomer, high temperature vulcanizing silicone rubber, and/or liquid silicone rubber (LSR) elastomer, to name a few. In embodiments, the elastomeric material has a tensile strength value of for example in the range of 0.5 to 15 MPa. In embodiments, the elastomeric material has a Young's modulus value of for example in the range of 0.0005 to 0.1 GPa. It is understood that the above ranges are merely exemplary and are not intended to be limiting, and the values may be less or more than the disclosed ranges. In embodiments, the mentum strap 110 is comprised of a tacky elastomeric material. In embodiments, the elastomeric material has a tackiness index. In embodiments, the material of the mentum strap 110, the tensile strength value and the Young's modulus value are selected and configured such that the mentum strap 110 is adequately flexible to conform to the shape of the mentum of the patient without significantly deforming or stretching.

As shown in FIG. 1, in embodiments, the mentum strap 110 further includes a plurality of perforations 116. As shown in FIG. 2, in embodiments, the mentum strap 110 comprises a central perforation 118. The perforations (116, 117) allow for the skin of the patient in contact with the first surface 112 of the mentum strap 110 to perspire and also reduces irritation of the skin due to accumulation of perspiration. During use, the patient's mentum may extend partially through the central perforation 118 so that the mentum strap 110 is held more securely in place.

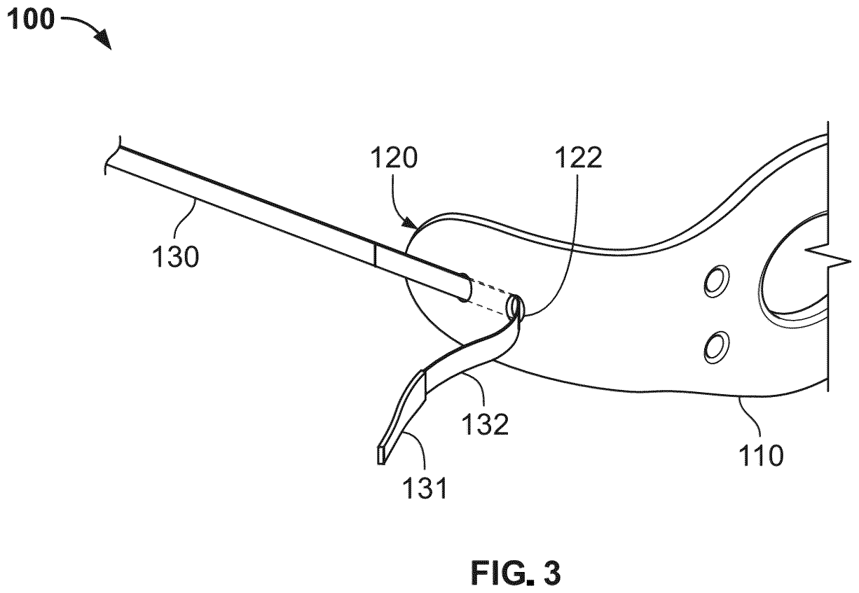

With reference to FIGS. 1 through 3, in embodiments, opposing distal portions 120 of the mentum strap 110 include attachment points 122 for attaching the pair of securement straps 130 to the respective attachment points 122. In embodiments, respective proximal portions 132 of the pair of securement straps 130 comprise a hook-and-loop portion 131 or other suitable attachment or securement configuration for removably attaching the respective proximal portions 132 of the pair of securement straps 130 to the attachment points 122 of the mentum strap 110. In embodiments, the pair of securement straps 130 are non-removably attached or otherwise secured to the mentum strap 110. For example, the pair of securement straps 130 may be sonic welded to the mentum strap 110 or otherwise adhered to the mentum strap 110. In embodiments, the mentum strap 110 and the pair of securement straps 130 are of a unitary body.

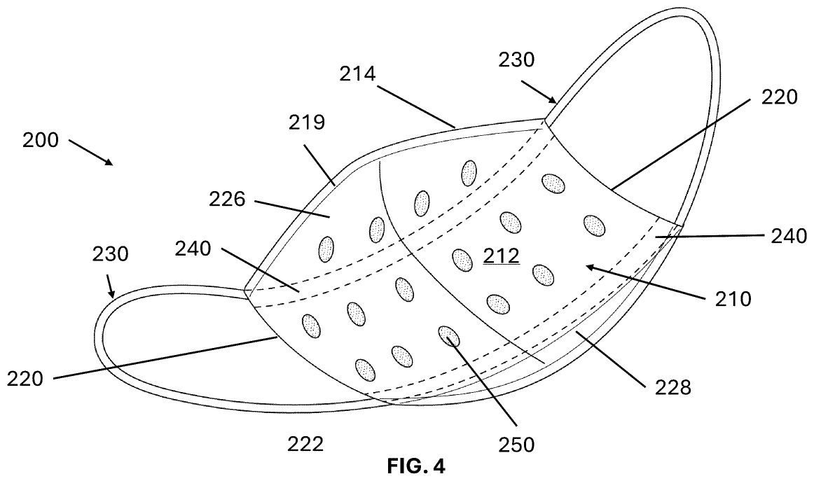

FIG. 4 shows a mentum securement device, generally designated by reference number 200, according to another exemplary embodiment of the present invention. The mentum securement device 200 includes a mentum strap 210 and a pair of securement straps 230 attached to the mentum strap 210. In embodiments, the mentum strap 210 has a shape, such as, for example, generally oval or a rectangular, to name just two. The mentum strap 210 has a first surface 212 and a second surface 214, and is defined by opposing distal portions 220, a superior end 226 and an opposing inferior end 228. In embodiments, the superior end 226 forms an extending portion of the mentum strap 210. The first surface 212 is configured to be positioned on and in a directly facing relation to a mentum of a patient undergoing anesthesia as explained in further detail below. The mentum strap 210 may have an irregular shape for better anatomical fit to the mentum of human.

In embodiments, the mentum strap 210 may be made of materials used in conventional medical or surgical masks, such as, for example, plastic, paper and nonwoven fabric, to name a few. The material may be fixed to one more malleable wires 219 so as to provide the mentum strap 210 with structural support and allow it to better conform to the mentum of a patient during use. For example, a malleable wire 219 may be disposed at the superior end 226 and/or the inferior end 228 of the mentum strap 210. The mentum strap 210 may include one or more pleats 240 to allow a user to adjust the mentum strap 210 from a flat configuration to a more cup-like configuration for better fit on the mentum of the patient. In embodiments, the first surface 212 of the mentum strap 210 may be at least partially coated with tacky material 250, such as, for example, foam, silicone and/or other types of polymeric materials, to name a few. The tacky material 250 may be applied in a pattern such as, for example, in a regular repeating pattern extending at least partially across the first surface 212 or in strips along one or more edges of the mentum strap 210.

In embodiments, the securement straps 230 may be elastics, and may be made of materials, such as, for example, fabric, polyester, spandex, elastodiene, or thermoplastic polyurethane (TPU), to name a few. Each securement strap 230 may be made of a single piece of material or more than one piece of material with the pieces of material configured for releasable attachment to one another (e.g., by tying or adhering distal end portions of the pieces together). In embodiments, the securement straps 230 are non-removably attached or otherwise secured to the mentum strap 210. For example, the pair of securement straps 230 may be sonic welded to the mentum strap 210 or otherwise adhered to the mentum strap 210. In embodiments, the mentum strap 210 and the pair of securement straps 230 are of a unitary body.

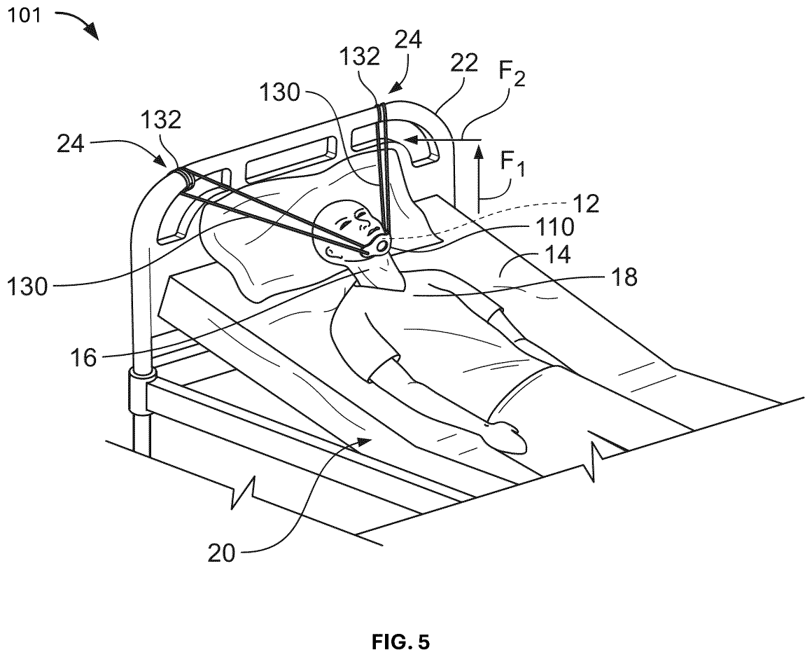

FIG. 5 is a perspective view of mentum securement system, generally designated by reference number 101, according to an exemplary embodiment of the present invention. The mentum securement system 101 includes the mentum securement device 100, 200 configured for securement to a mentum 12 of a patient 10 in accordance with exemplary embodiments of the present disclosure. As shown, the patient 10 is positioned on a surgical stretcher 20. In the illustrated embodiment, a portion of the surgical stretcher 20 is at an angle such that a head 14 and neck 16 of the patient 10 is resting at an angle relative to a torso 18 of the patient 10. In embodiments, the surgical stretcher 20 is flat such that the patient 10 rests in the supine position on the surgical stretcher 20 (as shown in FIG. 5C). It is understood that the surgical stretcher 20 is merely an exemplary embodiment of a resting surface for the patient 10 during a medical procedure requiring anesthesia. By way of example but not limitation, the surgical stretcher 20 may be an operating table, a split stretcher, or an emergency folding stretcher used in the field, and the like.

As shown, the mentum strap 110, 210 is secured to the mentum 12 of the patient 10 and distal portions 132, 232 of the pair of securement straps 130 are removably securable to a siderail 22 of the surgical stretcher 20. In embodiments, the siderail 22 of the surgical stretcher 20 is located at a position that is spaced from the head 14 of the patient 10 and is oriented perpendicularly to a length of the surgical stretcher 20. It is understood that the configuration of the siderail 22 is merely exemplary and is not intended to be limiting. By way of example, but not limitation, siderails may extend parallel to length of the surgical stretcher on either side of the patient 10, and the pair of securement straps 130 may be secured to each of the siderails.

The positions to which the distal portions 132 of the pair of securement straps 130 are secured to the siderail 22 define anchor points 24 of the surgical stretcher 20. In embodiments, the anchor points 24 may be a tie-down point integral to the surgical stretcher 20 or integral to the siderail 22. By way of example, the siderail 22 may include tie-down d-rings for securing the distal portions 134 of the pair of securement straps 130.

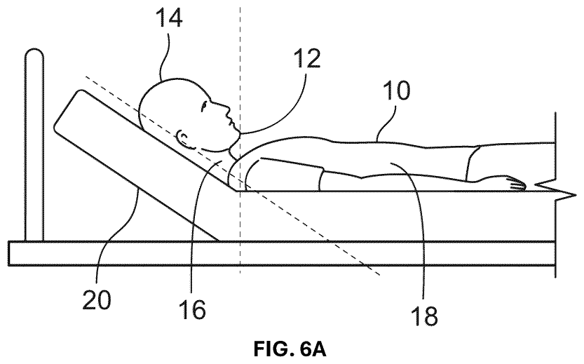

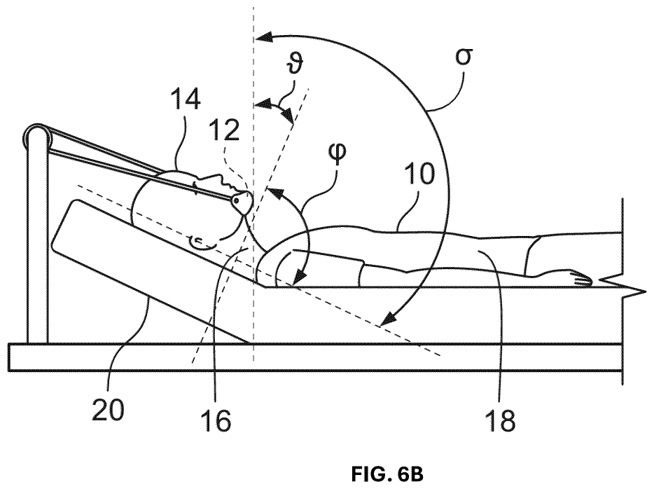

FIG. 6A is a side view of the patient 10 in a resting state where the mentum securement device 100 is not in use. As illustrated, the base of the head 14 is substantially planar with the neck 16 of the patient 10, both of which are resting supine on the surgical stretcher 20, defining a resting state of the mentum 12 of the patient 10. In the resting state, a resting angle φ is defined as the angle of the mentum 12 of the patient 10 relative to the neck 16 of the patient. As anesthesia is administered during a non-intubation anesthesia procedure, the airway may close resulting in labored breathing of the patient 10. A practitioner administering the anesthesia monitors the vital signs of the patient 10 (such as heartbeat, breathing rate, temperature, and blood pressure) while controlling the delivery of anesthesia to the patient. As the patient 10 is in the resting state as shown in FIG. 6A, the likelihood of an airway obstruction increases due to the angle of the mentum 12 of the patient relative to the neck 16 of the patient which causes the larynx to ovalize and narrow in cross-section or otherwise collapse. As the airway obstruction is occurring, the practitioner may have to intervene while continuing to monitor the breathing quality of the patient such that the airway remains open. This may last for a few minutes, or up to the remaining duration of the medical procedure. As a result, the practitioner may not be able to adequately monitor the vital signs of the patient 10 and control the delivery of anesthesia to the patient 10.

FIGS. 6B and 6C illustrate side views of the patient 10 with the mentum securement device 100 secured to the mentum 12 of the patient 10 and the distal portions 132 of the pair of securement straps 130 secured to respective anchor points 24. As shown, adjusting the length of the pair of securement straps 130 extends the mentum 12 of the patient 10 upward from the torso 16 of the patient. This results in the base of the head 14 of the patient 10 slightly lifting from the surgical stretcher 20, and the mentum 12 of the patient 10 being positioned in an extended state. The extended state is defined by the mentum 12 of the patient 10 being at an extended angle σ relative to the neck 12 of the patient. The extended angle σ is the sum of the resting angle φ and a tilt angle θ. Stated differently, the tilt angle θ is defined as the difference between the extended angle and the resting angle φ. Maintaing the mentum 12 of the patient 10 in the extended state results in straightening of the larynx and more generally the airway of the patient 10, thereby preventing, reducing or otherwise eliminating an airway obstruction. Stated differently, in the extended state, an otherwise obstructed airway of the patient 10 becomes unobstructed due to extending the mentum 12 of the patient by the tilt angle θ in the extended state. In embodiments, the tilt angle θ in the extended state is in the range of 10° to 15°, and may vary depending on the health of the patient, the amount of anesthesia or muscle relaxants are used, and the like. It is therefore understood that the range of 10° to 30° of the tilt angle θ is not intended to be limiting. By way of example, but not limitation, in patients who have pre-existing respiratory conditions due to disease or obesity may require the mentum 12 of the patient 10 to be extended at a tilt angle θ up to 30° or up to 35°. In embodiments, the tilt angle θ in the extended state is in the range of 10° to 35°.

Referring back to FIGS. 5 and 6B, to adjust the tilt angle, the practitioner may adjust the respective lengths of the securement straps 130 such that the securement straps 130 apply a tension force (illustrated as force components F₁ and F₂ in FIG. 4) to the mentum strap 110. In embodiments, each of the securement straps 110 include a webbing and buckle configuration to adjust the respective lengths of the pair of securement straps 130. In embodiments, each of the securement straps 110 includes a hook-and-loop configuration to adjust the respective lengths of the pair of securement straps 130 as explained in further detail below.

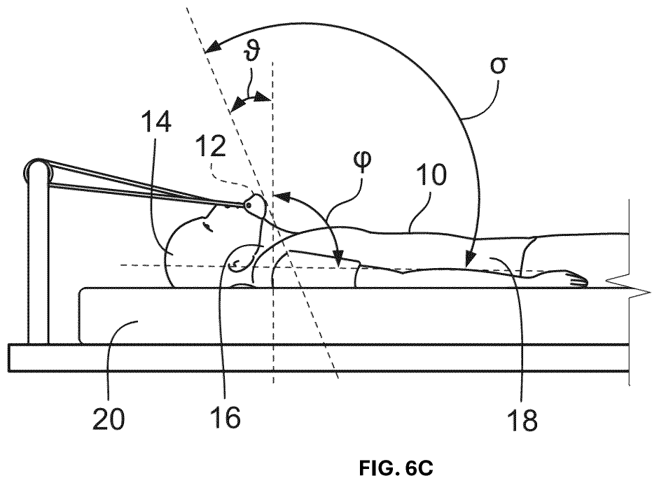

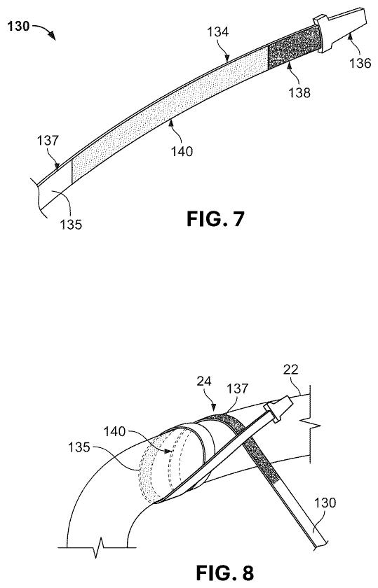

FIG. 7 is a front view of the distal portion 132 of a securement strap 130 and FIG. 8 is a perspective view of the distal portion 132 of the securement strap 130 attached to an anchor point 24 of the siderail 22 in accordance with exemplary embodiments of the present disclosure. As shown in FIGS. 7 and 8, in the embodiments, the distal portion 132 includes a distal pull-tab portion 136 adjacent to the distal portion 132. In embodiments, a first surface 135 of the securement strap 130 includes a hook-and-loop portion 138 (having a hook portion on the first surface 135 of the securement strap 130 and an opposing loop portion on a second surface 137 of the securement strap 130 as shown in FIG. 8) adjacent to the distal pull-tab portion 136. In embodiments, the first surface 135 further includes a tacky portion 138 adjacent to the hook-and-loop portion 136. Each of the hook-and-loop portion 136 and the tacky portion 138 are configured for removable securement of the distal portion 134 of the pair of securement straps 130 to the anchor point 124, and the distal pull-tab portion 136 is configured to release the distal portion 134 from the anchor point 124.

As shown in FIG. 8, the tacky portion 140 is wrapped around the siderail 22 and the hook portion of the hook-and-loop portion 138 on the first surface 135 is subsequently secured to the loop portion of the hook-and-loop portion 138 on the second surface 137, thereby removably securing the distal portion 132 of the securement strap 130 to the siderail 22 at the anchor point 24. In embodiments, the hook-and-loop portion is configured for adjustment of the respective lengths of the pair of securement straps 130 by interlocking the hook portion 136 to the loop portion 138 at one or more positions along lengths of the hook portion and loop portion, thereby increasing or decreasing the lengths of the securement strap 130.

To prevent overextension of the mentum 12 of the patient 10, in embodiments, the pair of securement straps 130 are configured to fail or snap upon application of excessive tension forces. In embodiments, the excessive tension force is in the range of 12-15 lbs-ft. In embodiments, the tension force is selected such that the tilt angle θ in the extended state cannot exceed the above referenced ranges of 10° to 35° relative to the resting state of the mentum 12. In embodiments, the attachment points 122 of the mentum strap 110 may be configured to fail upon application of the excessive tension forces. In embodiments, the hook-and-loop portion 131 of the proximal portion 132 of the securement strap 130 or the hook-and-loop portion 136 of the distal portion 134 of the securement strap 130 may be configured to fail or otherwise detach upon application of the excessive tension forces.

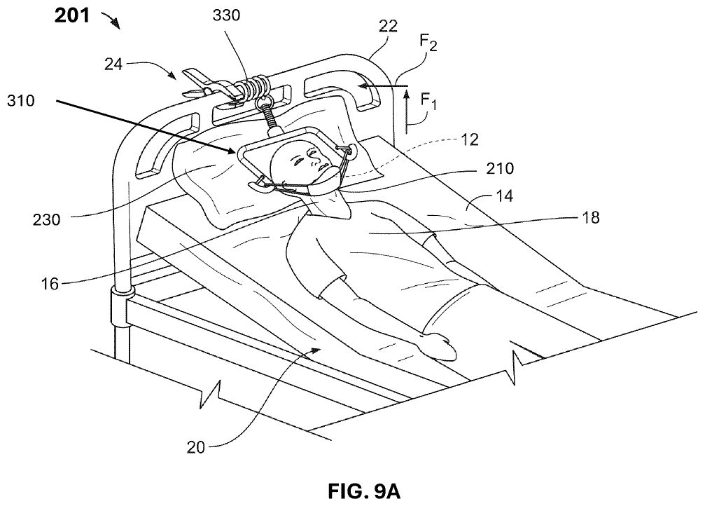

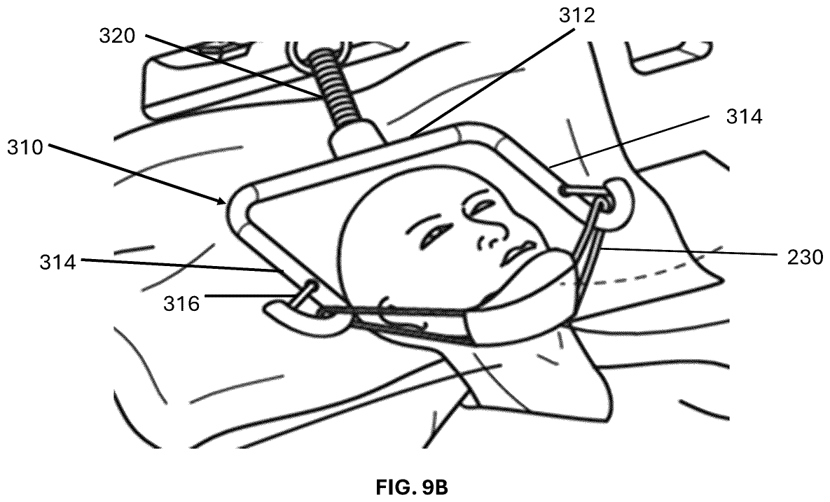

FIG. 9A is a perspective view of the mentum securement system, generally designated by reference number 201, according to an exemplary embodiment of the present invention. The mentum securement system includes the mentum securement device 200 configured for securement to a mentum 12 of a patient 10 in accordance with exemplary embodiments of the present invention. As shown, the patient 10 is positioned on a surgical stretcher 20. In the illustrated embodiment, a portion of the surgical stretcher 20 is at an angle such that a head 14 and neck 16 of the patient 10 is resting at an angle relative to a torso 18 of the patient 10. In embodiments, the surgical stretcher 20 is flat such that the patient 10 rests in the supine position on the surgical stretcher 20 (as shown in FIG. 6C). It is understood that the surgical stretcher 20 is merely an exemplary embodiment of a resting surface for the patient 10 during a medical procedure requiring anesthesia. By way of example but not limitation, the surgical stretcher 20 may be an operating table, a split stretcher, or an emergency folding stretcher used in the field, and the like.

The mentum securement device 200 may be attached to a brace 310. As shown more clearly in FIG. 9B, the brace 310 may be generally U-shaped and include a first portion 312 and two second portions 314 extending perpendicular relative to the first portion 312. The brace 310 is configured so that it may be placed over the patient's head with the two second portions 314 extending lengthwise on either side of the head and the first portion 312 extending cross-wise above the patient's head. The brace 310 may be made of two or more separate components or may be a unitary structure. In exemplary embodiments, the brace 310 is made of materials, such as, for example, plastic, metal and ceramic, to name a few.

As shown in FIG. 9B, each of the securement straps 230 may be attached to a respective one of the two second portions 314 of the brace 310. In exemplary embodiments, the brace 310 may include clips 316 that assist in securing the securement straps 230 to the two second portions 314. The clips may be spring-loaded. The end portions of the two second portions 314 may be shaped to prevent the securement straps 230 from detaching from the brace 310. For example, as shown in the figures, the end portions of the two second portions 314 may be bent or otherwise shaped to avoid inadvertent detachment of the mentum securement device 200.

In exemplary embodiments, the brace 310 is attached to an adjustable spring mechanism 320, which in turn is attached to a clamping mechanism 330. The adjustable spring mechanism 320 may be, for example, a tension spring that is adjustable by lengthening or shortening the distance between the brace 310 and the clamping mechanism 330. For example, the tension spring may include a screw mechanism that can be turned to shorten or lengthen the spring. In this regard, turning the screw in a first direction may lengthen the spring to increase pulling force while turning the screw in the opposite direction may shorten the spring to decrease the pulling force. In exemplary embodiments, the clamping mechanism 330 may be, for example, a spring clamp, a quick action clamp, a C-clamp, an F-clamp, or a screw clamp, to name a few. The clamping mechanism 330 is configured for attachment to a portion of a surgical stretcher 20, such as, for example, a side rail 22.

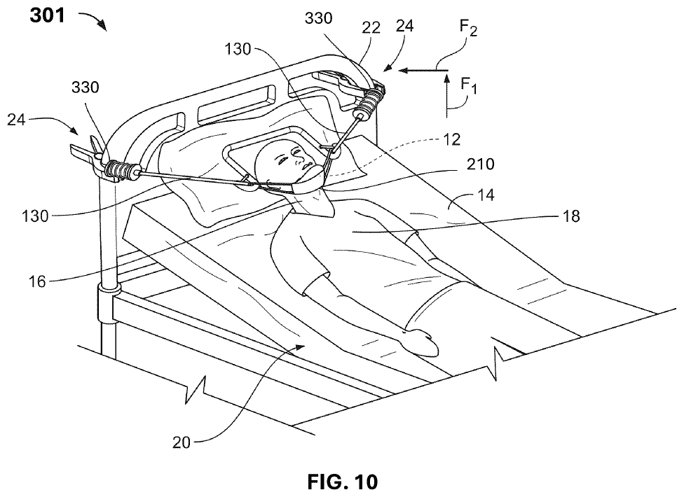

Although FIG. 9A shows just one adjustable spring mechanism 320 and one corresponding clamping mechanism 330, it should be appreciated that the mentum securement device 200 may be attached to the surgical stretcher using any number of spring mechanisms 320 and clamping mechanisms 330. For example, in the mentum securement system 301 shown in FIG. 10, the mentum securement device 200 may be attached using two adjustable spring mechanisms 320-1, 320-2 and two corresponding clamping mechanisms 330-1, 330-2. Each adjustable spring mechanism 320-1, 320-2 may be attached to a corresponding side of the brace 310.

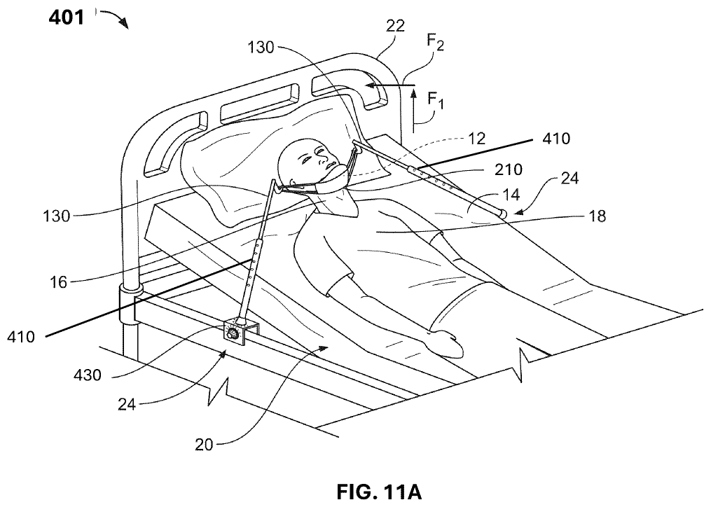

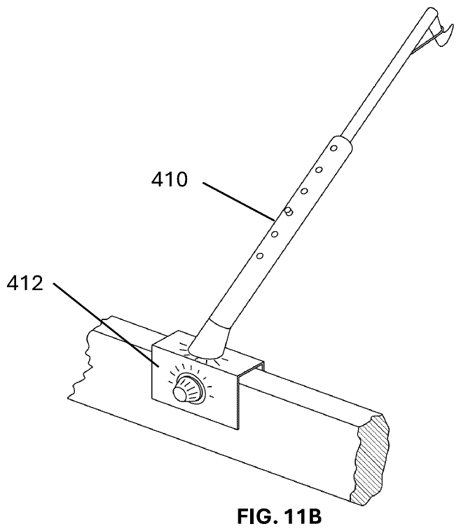

FIG. 11 is a perspective view of a mentum securement system, generally designated by reference number 401, according to an exemplary embodiment of the present invention. The system 401 includes the mentum securement device 200 configured for attachment to a mentum 12 of a patient 10 in accordance with exemplary embodiments of the present invention. As shown, the patient 10 is positioned on a surgical stretcher 20. In the illustrated embodiment, a portion of the surgical stretcher 20 is at an angle such that a head 14 and neck 16 of the patient 10 is resting at an angle relative to a torso 18 of the patient 10. In embodiments, the surgical stretcher 20 is flat such that the patient 10 rests in the supine position on the surgical stretcher 20 (as shown in FIG. 5C). It is understood that the surgical stretcher 20 is merely an exemplary embodiment of a resting surface for the patient 10 during a medical procedure requiring anesthesia. By way of example but not limitation, the surgical stretcher 20 may be an operating table, a split stretcher, or an emergency folding stretcher used in the field, and the like.

The mentum securement device 200 may be attached to the surgical stretcher 20 by rigid rods 410. The length of the rods 410 may be adjustable. In this regard, the rods 410 may have telescoping portions that are adjustable relative to one another using length adjustment mechanisms, such as, for example, detent mechanisms, screws, or threaded attachment between the telescoping portions, to name a few. Distal end portions of the rods 410 are attached to the stretcher 20 by clamping mechanisms 430 while proximal end portions of each rod 410 are attached to a corresponding one of the securement straps 230. In this regard, the proximal end portions of the rods 410 may include clips to aid in attachment to the securement straps 430. As shown more clearly in FIG. In exemplary embodiments, the clamping mechanisms 430 allow for angular adjustment of the rods 410 using, for example, a lockable pivot joint 412. The angular adjustment allows the rods 410 to be positioned so as to apply a pushing force, thereby pivoting the patient's head upwards to open the patient's airway.

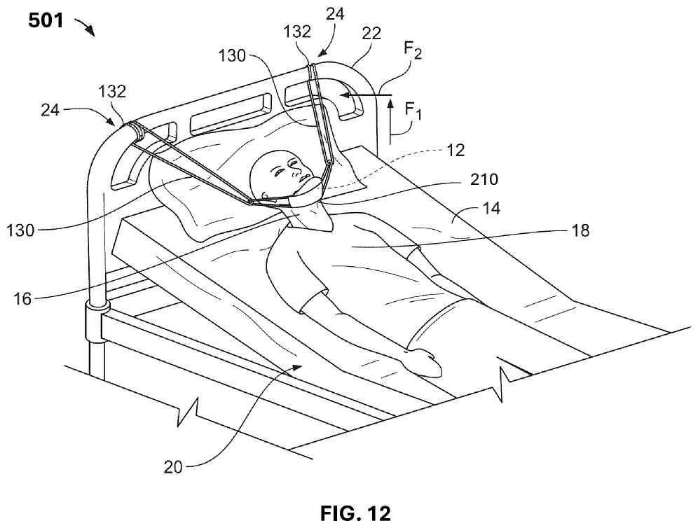

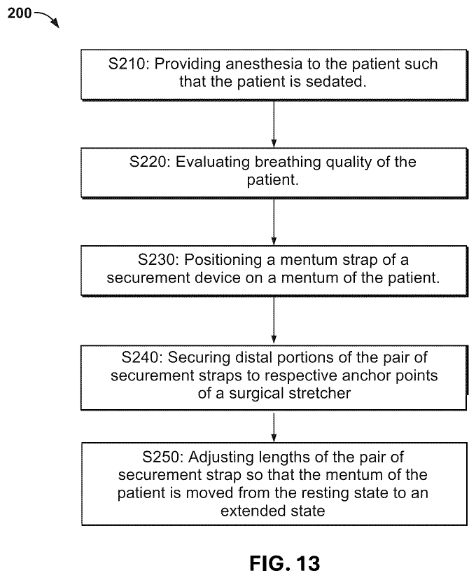

FIG. 12 is a perspective view of a mentum securement system, generally designated by reference number 501, according to an exemplary embodiment of the present invention. The mentum securement system 501 is generally the same as the system shown in FIG. 5 except that the mentum securement device 200 is used in the place of the mentum securement device 100. Also, although FIG. 12 shows a looped strap attached to each of the securement straps 230, it should be appreciated that the straps need not be looped around the anchor point but instead may be attached to the anchor point by, for example, a hook-and-loop attachment or a buckle mechanism, so that the straps are arranged in a non-looping manner between the mentum securement device 200 and the anchor point. FIG. 13 illustrates steps for a method 200 of preventing obstruction of an airway passage of a patient undergoing an anesthetic procedure. The method 200 includes providing S210, by a practitioner, anesthesia to the patient such that the patient is sedated. As the patient is sedated, the method further includes evaluating S220, by the practitioner, breathing quality of the patient. In embodiments, the breathing quality of the patient is evaluated by detecting one or more of labored breathing, obstructed breathing, and snoring. In embodiments, the breathing quality of the patient is evaluated by monitoring carbon dioxide readings from a gas sampling device (not shown) attached to the patient. The method 200 further includes positioning S230, by the practitioner, a mentum strap of a securement device on a mentum of the patient. The method further includes securing S240, by the practitioner, distal portions of the pair of securement straps to respective anchor points of a surgical stretcher. As discussed herein, the securement straps may be attached directly to anchor points of the stretcher, attached to a brace which is in turn attached to the anchor points by an adjustable spring mechanism and/or attached to adjustable rods which are in turn attached to the anchor points. In step S250, lengths of the pair of securement strap are adjusted by the practitioner (e.g., by adjusting the spring mechanism or by adjusting length and/or angle of the rods) so that the mentum of the patient is moved from the resting state to an extended state, wherein in the extended state the mentum of the patient is at an extended angle relative to a neck of the patient and the patient no longer has obstructed breathing, wherein the extended angle is greater than the resting angle.

While this invention has been described in conjunction with the exemplary embodiments outlined above, it is evident that many alternatives, modifications and variations will be apparent to those skilled in the art. Accordingly, the exemplary embodiments of the invention, as set forth above, are intended to be illustrative, not limiting. Various changes may be made without departing from the spirit and scope of the invention.

Now that embodiments of the present disclosure have been shown and described in detail, various modifications and improvements thereon can become readily apparent to those skilled in the art. Accordingly, the exemplary embodiments of the present disclosure, as set forth above, are intended to be illustrative, not limiting. The spirit and scope of the present disclosure is to be construed broadly.

What is claimed is:

1. A mentum securement device comprising:
a mentum strap defined by opposing distal portions, a superior end and an opposing inferior end, and a first surface configured to be positioned on and in facing relation to a mentum of a patient undergoing anesthesia; and,
two or more securement straps each having a proximal portion attached to a respective opposing distal portion of the mentum strap and a distal portion configured for securement to an anchor point,
wherein the mentum strap and the two or more securement straps are configured to position the mentum of the patient to an extended state, wherein in the extended state the mentum of the patient is at an extended angle relative to a neck of the patient, wherein in a resting state the mentum of the patient is at a resting angle relative to a neck of the patient, wherein the extended angle is greater than the resting angle.

2. The mentum securement device of claim 1, wherein an airway passage of the patient is unobstructed in the extended state.

3. The mentum securement device of claim 2, wherein a tilt angle is defined as the difference between the extended angle and the resting angle, and the tilt angle in the extended state is in the range of 10° to 30°.

4. The mentum securement device of claim 3, wherein the tilt angle is set by adjustment of a respective length of the two or more securement straps.

5. The mentum securement device of claim 1, wherein each of the two or more securement straps comprise a webbing and buckle configuration to adjust the respective lengths of the two or more securement straps.

6. The mentum securement device of claim 5, wherein the two or more securement straps further comprise a hook-and-loop portion at the distal portion configured for adjustment of the respective lengths of the two or more securement straps by interlocking the hook-and-loop portion at one or more positions along lengths of the two or more securement straps.

7. The mentum securement device of claim 1, wherein each of the distal portions of the two or more securement straps comprise a hook-and-loop portion configured to removably secure the distal portions to respective anchor points in a hook-and-loop securement.

8. The mentum securement device of claim 7, wherein the respective anchor point is a portion of a siderail of a surgical stretcher.

9. The mentum securement device of claim 7, wherein the respective anchor point is a tie-down d-ring of a surgical stretcher.

10. The mentum securement device of claim 1, wherein each of the distal portions of the two or more securement straps comprise a tacky first surface to removably secure the distal portions to respective anchor points.

11. The mentum securement device of claim 1, wherein the two or more securement straps are configured to fail upon application of a predetermined tension force.

12. The mentum securement device of claim 1, wherein the mentum strap is comprised of an elastomeric material.

13. The mentum securement device of claim 12, wherein the elastomeric material comprises a material selected from the group consisting of: medical grade silicone, heat cured rubber (HSR) elastomer, high temperature vulcanizing silicone rubber, and liquid silicone rubber (LSR) elastomer.

14. The mentum securement device of claim 1, wherein at least a portion of the first surface comprises a tacky surface configured to be removably adhered to the mentum of the patient.

15. The mentum securement device of claim 14, wherein the first surface comprises an adhesive coating.

16. The mentum securement device of claim 1, wherein the mentum strap is comprised of a tacky elastomeric material.

17. The mentum securement device of claim 1, wherein the mentum strap further comprises a plurality of perforations.

18. The mentum securement device of claim 1, wherein the mentum strap comprises one or more pleats.

19. The mentum securement device of claim 1, further comprising a brace comprising a proximal end portion configured for attachment to the two or more securement straps.

20. The mentum securement device of claim 19, wherein the brace further comprises a distal end portion configured for attachment to the anchor point.

21. The mentum securement device of claim 20, further comprising a clamping mechanism configured for attachment to the distal end portion of the brace and the anchor point.

22. The mentum securement device of claim 21, further comprising an adjustable tension device disposed between the clamping mechanism and the brace.

23. The mentum securement device of claim 1, further comprising two or more rods each comprising a proximal end portion configured for attachment to a corresponding one of the two or more securement straps and a distal end portion configured for attachment to respective anchor points.

24. The mentum securement device of claim 23, wherein the two or more rods are adjustable in length.

25. The mentum securement device of claim 24, wherein an angle at which the two or more rods extend relative to the respective anchor points is adjustable.
